(19) United States
(12) Patent Application Publication
(10) Pub. No.: US 2004/0092011 A1
(43) Pub. Date: May 13, 2004

(54) Adipocytic differentiated adipose derived adult stem cells and uses thereof

(76) Inventors: William O. Wilkison, Bahama, NC (US); Yuan-Di C. Halvorsen, Branford, CT (US); Jeffrey M. Gimble, Baton Rouge, LA (US); William Franklin, Morrisville, NC (US)

Correspondence Address: KATHRYN DOYLE , ESQ., MORGAN & LEWIS , LLP, 1701 MARKET STREET, PLILADELPHIA, PA, 19103, US

(21) Appl. No.: 10/406,479

(22) Filed: Apr. 3, 2003

Related U.S. Application Data
(60) Provisional application No. 60/370,089, filed on Apr. 3, 2002.

(51) Int. Cl. C12N 5/08

(52) U.S. Cl. 435/366

(57) ABSTRACT

The present invention is differentiated adipose tissue-derived stromal cells that exhibit the improved properties of increased extracellular matrix proteins and/or a lower amount of lipid than a mature isolated adipocyte. Methods for the expansion and differentiation of these cells are also provided. The cells of the invention are used for the treatment, repair, correction and/or regeneration of soft tissue cosmetic defects.

### CROSS REFERENCE TO RELATED APPLICATION

This application claims priority to U.S. Provisional Application No. 60/370,089 filed on Apr. 4, 2002.

### FIELD OF INVENTION

This invention is for compositions and methods for the differentiation of adipose derived adult stem cells into adipocytes and uses thereof.

### BACKGROUND OF INVENTION

Adipose Tissue Biology

Adipose tissue plays an important and overlooked role in the normal development and physiology of humans and other mammalian species. Many different kinds of fat exist. The most common type is white adipose tissue, located under the skin (subcutaneous fat), within the abdominal cavity (visceral fat) and around the reproductive organs (gonadal fat). Less common in the adult human is brown adipose tissue, which plays an important role in generating heat during the neonatal period; this type of fat is located between the shoulder blades (interscapular), around the major vessels and heart (periaortic and pericardial), and above the kidney (suprarenal). As women mature, they develop increased amounts of mammary adipose tissue. The mammary fat pad serves as an energy source during periods of lactation. Indeed, reproductive capacity and maturation are closely linked to the adipose tissue stores of the individual. Puberty in women and men correlates closely with the production and release of leptin, an adipose tissue derived hormone, and to body fat composition. Other adipose tissue sites play a structural role in the body. For example, the mechanical fat pads in the soles of the feet provide a cushion against the impact of walking. Loss of this fat depot leads to progressive musculoskeletal damage and impaired mobility. Bone marrow fat cells are present in bone marrow to provide energy to developing blood cells within the marrow. Bone marrow adipocytes are different than adipocytes present in adipose tissue, differing in morphology, physiology, biochemistry as well as their response to various stimulators such as insulin. Adipocytes present in bone marrow stroma may function to: 1) regulate the volume of hemodynamically active marrow; 2) serve as a reservoir for lipids needed in marrow cell proliferation, and 3) may be developmentally related to other cell lineages such as osteoblasts. White adipose tissue (i.e. body fat) in contrast, is involved in lipid metabolism and energy homeostasis. [(Gimble, “The Function of Adipocytes in the Bone Marrow Stroma”, The New Biologist 2(4), 1990, pp. 304-312).]

Adipose-Related Diseases

Obesity is currently the major disorder affecting people of all ages in the United States and other countries where calorie-rich diets and a sedentary lifestyle are common. Nevertheless, there are a significant number of individuals who are afflicted by conditions or diseases, which result from an absence of adipose tissue. Lipodystrophy, either partial or generalized, is a potentially life-threatening illness and the most severe example. This disorder is most common in women and is characterized by a loss of subcutaneous adipose tissues. The results can be disfiguring. The nature of the disease remains poorly understood. In some families, there is a strong genetic component with evidence of an autosomal trait [Peters J M, Barnes R, Bennett L, Gitomer W M, Bowcock A M, Garg A Nat Genet 18:292-295, 1998; Jackson S N, Pinkney J, Bargiotta A, Veal C D, Howlett T A, McNally P G, Corral R, Johnson A, Trembath R C Am J Hum Genet 63:534-540, 1998; Garg A, Wilson R, Barnes R, Arioglu E, Zaidi Z, Gurakan F, Kocak N, O'Rahilly S, Taylor S I, Patel S B, Bowcock A M J Clin Endocrinol Metab 84:3390-3394, 1999] Some cases may represent an autoimmune disease, since patients display abnormalities in their complement system.

Other cases appear to be the consequence of medication. Patients receiving HIV-1 protease inhibitors exhibit peripheral lipodystrophy [Carr A, Samaras K, Chisholm D J, Cooper D A Lancet 351:1881-1883, 1998]. Lipodystrophy is not simply a cosmetic defect. Patients suffering from this disease also exhibit a complex metabolic dysregulation characterized by hyperlipidemia, hyperinsulinemia, and insulin resistant diabetes [Jackson et al Am J Hum Genet 63:534-540, 1998]. Because these patients respond poorly to insulin, their diabetes and its inherent complications cannot be controlled by conventional therapies. While the new oral antidiabetic thiazolidinedione drugs offer a therapeutic option, additional treatments will be necessary.

Recently described animal models of lipodystrophy may point to the direction such treatments. Two different transgenic mice have been created where adipose tissue sites are greatly depleted [Shimomura I, Hammer R E, Richardson J A, Ikemoto S, Bashmakov Y, Goldstein J L, Brown M S Genes Dev 12:3182-3194, 1998; Moitra J, Mason M M, Olive M, Krylov D, Gavrilova O, Marcus-Samuels B, Feigenbaum L, Lee E, Aoyama T, Eckhaus M, Reitman M L, Vinson C 12:3168-3181, 1998]. In both sets of animals, this is accompanied by diabetes, >50-fold elevation in insulin levels, elevated serum triglycerides and free fatty acids. These animals demonstrate that the absence of fat can cause diabetes. Preliminary studies indicate that these mice can be successfully treated by implantation of exogenous adipose tissue from a histocompatibility donor. The same may be true for humans.

Soft Tissue Cosmesis

There are non-life threatening human conditions associated with an absence or loss of adipose tissue. While these disorders are cosmetic, they have a significant impact on quality of life issues in afflicted individuals. For example, facial acne in adolescents can result in the loss of subcutaneous adipose tissue and severe disfiguring. Similar soft tissue scarring and defects can occur in patients receiving radiation as an adjunctive treatment for cancer. These can be treated by transplantation of the patient's own fat into the afflicted area [Billings E, May Plast Reconstruct Surg 83:368-381,1989;J W Bircoll M, Novack B Ann Plast Surg 18:327-329, 1987; Ellenbogen R Ann Plast Surg 16:179-194, 1986]. However, the success of these repairs is often transient due to resorption of the transplanted cells by the surrounding tissue [Ersek R A Plast Reconstruct Surg 87:219-227,1991].

Some efforts to improve this have involved the incorporation of dextran beads adsorbed with basic fibroblast growth factor [Eppley B L, Sidner R A, Platis J M, Sadove A M Plast Reconstruct Surg 90:1022-1030, 1992]. Scars due to injury or surgery may have similar sequelae. The most extreme cases are those due to the treatment of life-threatening breast cancer, requiring either a lumpectomy or mastectomy. Some patients choose an artificial breast implant as a cosmetic option. Many prefer to undergo an extensive surgical transplantation of their abdominal adipose tissue and musculature to repair any disfigurement. These “TRAM flap” surgeries must be performed at the time of the mastectomy and cannot be repeated. Their success rate is variable. Impaired vascular supply to the transplanted tissue can lead to failure and resorption of the transplanted fat tissue. Alternative repair processes with improved vascularization and reduced necrosis of the adipose tissues would benefit these individuals and potentially reduce their medical expenditures.

During the past few years, there have been significant advancements in the field of biomaterials. Many materials are currently available to treat these conditions. The most commonly used product is bovine collagen, which can be injected into wrinkles or acne scars to act as a “filler” Other naturally occurring materials are being developed for use in treating cosmetic defects, including human collagen. Many biologically occurring matrices and lattices have been shown to support the growth of tissue and cells. New matrices for the engineering of tissues ex vivo have been developed. These include the use of biocompatible, compounds including poly-lactic acid, poly-glycolic acid, collagen type I derivatives and alginate, among others. It is possible to incorporate exogenous factors into these materials to facilitate the ingrowth of host cells or to accelerate the growth of donor cells implanted into the matrix. These exogenous factors can include chemical compounds, such as steroids, or DNA compounds, such as plasmids encoding a genetically engineered cytokine or growth factor capable of accelerating neovascularization of the matrix itself. In one recent study, new adipose tissue was created in mice following the injection of basement membrane collagen (Matrigel) together with basic fibroblast growth factor [Kawaguchi N, Toriyama K, Nicodemou-Lena E, Inou K, Torii S, Kitagawa Y Proc Natl Acad Sci USA 95:1062-1066, 1998]. The injection site was characterized by new blood vessel formation and the migration of host adipocyte precursor cells into the exogenous matrix. Additional studies in rats have incorporated adipose tissue derived stem cells into poly lactic-co-glycolic disks and observed the formation of mature adipocytes when these devices are implanted into recipients [Patrick C W, Chauvin P B, Hobley J, Reece G P Tissue Eng 5:139-151, 1999].

Despite these advancements, there have not been any successful breakthroughs in the generation of implantable adipose or soft tissues in man. One of the major limitations has been a failure to develop optimal conditions for the proliferation, expansion, and differentiation of human adipocytes or fat cells ex vivo and the development of optimal conditions for the successful transplantation of autologous or allogeneic adipocytes in man. Most currently used procedures require surgeons to harvest the donor fat and re-implant it into a distant site within a single operation. The procedures for the handling and care of the adipose tissue result in significant cell damage and death, ultimately leading to resorption and cosmetic failure of the surgery over time [Ersek R A Plast Reconstruct Surg 87:219-227,1991]. Furthermore, adipocytes that have secreted extracellular matrix may provide a better starting point for creating adipose tissue in vivo for some of the arguments presented above. The nascent matrix secreted by an adipocyte is likely the most favorable materials for creating adipose tissue.

What is needed is the combination of a variety of biomaterials with cell-based technology to allow for the preparation of bioengineered adipocytes to treat any and all conditions involving an absence or loss of soft tissue depots within the body.

Therefore, an object of the invention is to provide cell-based compositions and methods for use in tissue cosmesis, tissue repair, and for the treatment of such fat-related disorders as lipodystrophy.

### SUMMARY OF THE INVENTION

The invention provides an adipose tissue-derived adult stem cell that is differentiated to express at least one characteristic of an adipocyte and which exhibits one or more properties that are superior to known naturally occurring adipocytes. In one embodiment, the cell contains a substantially greater amount of extracellular matrix proteins than a mature isolated adipocyte. In another embodiment, the cell contains a significantly greater amount of extracellular matrix proteins than a mature isolated adipocyte. Since the primary protein in the extracellular matrix is collagen, the cell of the invention will contain a larger quantity of collagen than that in isolated mature adipocytes or adipocytes produced by prior art methods.

The differentiated cell also, or in the alternative, contains a substantially lower amount of lipid than a mature isolated adipocyte. In another embodiment, the cell contains a significantly lower amount of lipid. The differentiated cell of the invention exhibits oil droplets that are smaller in diameter than those of either mature isolated adipocytes or those produced under other culturing and differentiation procedures. The cell of the invention has the advantage of greater stability during culturing because it is less likely to detach from cultureware, float to the surface of culture media or be lost during subsequent feeding. The differentiated cell is also more resistant to mechanical damage or shearing force during cell harvesting and implanting procedures.

The adipose tissue-derived stromal cell used in the new differentiation process can come from any animal but in a preferred embodiment is human. The differentiated cell of the invention can also be modified with a nucleic acid, which in one embodiment, allows the insertion of a chemical probe.

Another aspect of the invention is a method for expanding the growth of an isolated adipose tissue-derived stem cell comprising: (a) plating the cells at varying densities in a growth maintenance medium comprising a chemically defined cell culture medium without enzymatic digestion and re-plating of the cells; and then (b) incubating the cells for long cell growth periods in a single cultureware flask to optimize the production of extracellular matrix. The invention provides the significant advantage over previous methods in that cells are grown in a single cultureware flask for long periods of time eliminating multiple passages of the cultures during the initial isolation and culturing thus improving efficiency, increasing yield, and decreasing cost.

Still another aspect of the invention is a method for differentiating an adipose tissue-derived stem cell into a cell that possesses at least one characteristic of an adipocyte, comprising an adipocyte differentiation medium comprising a defined cell culture medium having or supplemented with a concentration of a peroxisome proliferator-activated receptor gamma (PPARγ) agonist that is not a thiazolidinedione and is preferably indomethacin or an indomethacin derivative.

Another aspect of the invention is a method for differentiating an adipose tissue-derived stem cell into a cell that possesses at least one characteristic of an adipocyte, comprising incubating the cells for long cell growth periods in a single cultureware flask to optimize the production and size of lipid vacuoles.

Still another aspect of the invention is a method for differentiating an adipose tissue-derived stem cell into a cell that possesses at least one characteristic of an adipocyte, comprising: plating the cells at varying densities in a growth maintenance medium comprising a chemically defined cell culture medium without enzymatic digestion and re-plating of the cells.

In still another aspect of the invention is a method for differentiating an adipose tissue-derived stem cell into a cell that possesses at least one characteristic of an adipocyte, comprising:

(a) plating the cells at varying densities in a growth maintenance medium comprising a chemically defined cell culture medium without enzymatic digestion and re-plating of the cells;

(b) incubating the cells for long cell growth periods in a single cultureware flask to optimize the production of extracellular matrix; (c) replacing the growth maintenance medium with an adipocyte differentiation medium comprising a defined cell culture medium having or supplemented with a concentration of a peroxisome proliferator-activated receptor gamma (PPARγ) agonist that is not a thiazolidinedione and is preferably indomethacin or an indomethacin derivative; (d) incubating the cells; (e) replacing the adipocyte differentiation medium with a growth maintenance medium; and then (f) incubating the cells for long cell growth periods in a single cultureware flask to optimize the production and size of lipid vacuoles.

The cells of the invention can be implanted into a host of any species. The host can be normal, diseased or immunodeficient. The host can be the same species as that which the adipose stem cell was derived from or can be another species.

The cell of the invention optionally is labeled with a probe that is in one embodiment adenoviral, retroviral, or fluorescent.

Yet another aspect of the invention is a method for developing three dimensional biomaterial matrices containing the adipose tissue-derived stem cells described herein, wherein the cells are capable of generating an adipose tissue depot upon implantation into a host recipient. The biomaterial matrices can be structured from any known biocompatible material including but not limited to poly-lactic acid, poly-glycolic acid, alginate, and a collagen type derivative.

Furthermore, the method includes a chemical inducing factor. The chemical inducing factor can comprise a protein, lipid, carbohydrate, polypeptide, nucleic acid or hormone. These can include a cyclic AMP inducer such as isobutylmethylxanthine, a glucocorticoid or glucocorticoid analogue such as dexamethasone, insulin or an insulin analogue, a peroxisome proliferator-activated receptor gamma agonist such as indomethacin or an indomethacin derivative. Additional agents optionally include but are not limited to cytokine or growth factor proteins such as fibroblast growth factor, vascular endothelial growth factor or bone morphogenetic proteins, and/or plasmids or other recombinant DNA vectors containing the cDNAs encoding such growth factors or proteins and incorporating biopolymers such as polylactic acids, poly-glycolic acids, hyaluronates, derivatives of glycosaminoglycans or derivatives of collagen.

The chemical inducing factor enhances the adherence, growth, differentiation, proliferation, vascularization and three-dimensional modeling of adipose tissue-derived stem cells into a soft tissue or adipose tissue depot either in vivo or ex vivo.

The present invention also provides methods for determining the ability of these culture conditions to direct the differentiation and function of these cells into adipocytes ex vivo and for the ability of these implants to differentiate and function physiologically upon implantation into a living organism.

The cell of the invention has use in drug discovery for compounds and proteins with relevance to human conditions involving adipocytes and for the direct treatment and repair of disease states such as lipodystrophy and disfiguring scars secondary to trauma, acne, or surgery for breast cancer and other life threatening diseases.

### DETAILED DESCRIPTION OF THE INVENTION

The present invention provides methods and compositions for the consistent and quantitative differentiation of stem cells isolated from adipose tissue into a cell that possesses at least one genotypic or phenotypic characteristic of an isolated primary adipocyte that is superior to an isolated mature adipocyte or an adipocyte produced by any other prior art method and their incorporation into a biomaterial matrix suitable for implantation and the subsequent development of a de novo adipose tissue depot.

I. Definitions

By “isolated mature adipocyte” is meant a fully differentiated adipocyte or population of adipocytes that are directly isolated from an animal.

“Adipocyte produced by any other method” or “produced by any other prior art method” refers to a fully differentiated adipocyte or cell with an adipocytic characteristic that is produced by a method or procedure not disclosed herein.

“By smaller amount of oil droplets” or “smaller amount of fat” means cells in which the observed oil droplets or lipid-contain vacuoles are significantly or substantially smaller than in isolated mature adipocytes or adipocytes produced by any other prior art method.

“Developmental phenotype” is the potential of a cell to acquire a particular physical phenotype through the process of differentiation.

“Hormone” is any substance that is secreted by a cell and that causes a phenotypic change in the same or another cell upon contact.

“Genotypic” is expressing at least one messenger RNA transcript of a gene associated with a differentiation pathway.

“Autoimmune disease” is intended to encompass any immune mediated process, humoral or cellular, that results in the rejection and destruction of the hosts' end organ. The etiology of this process is, but is not limited to, an immune response to an infection by an agent such as a virus, inborn metabolic propensity to autoimmune dysfunction, or a chemical exposure.

By “biomaterial matrices” is meant any biocompatible compound, resorbable or non-resorbable, which is able support the adherence, growth, differentiation, proliferation, vascularization, and three-dimensional modeling of adipose tissue-derived stem cells into a soft tissue or adipose tissue depot either in vivo or ex vivo. These may include, but are not limited to, poly-lactic acid, poly-glycolic acid, hyaluronates, derivatives of glycosaminoglycans, alginate, collagen type I and its derivatives, collagen type IV and its derivatives, any other collagen type and its derivatives, and any combination thereof.

By “chemical inducing factors” is meant any chemical agent, either protein, lipid, or carbohydrate in character, which enhances the adherence, growth, differentiation, proliferation, vascularization and three-dimensional modeling of adipose tissue-derived stem cells into a soft tissue or adipose tissue depot either in vivo or ex vivo. These include but are not limited to monobutyrin, thiazolidinediones, glucocorticoids, and long chain fatty acids all at concentrations including, but not limited to, 10⁻⁹ to 10⁻³ molar. The preferred compound is indomethacin or an indomethacin derivative.

By “protein growth factors and cytokines” is meant any protein hormone, growth factor, or cytokine which enhances the adherence, growth, differentiation, proliferation, vascularization, and three-dimensional modeling of adipose tissue-derived stem cells into a soft tissue or adipose tissue depot either in vivo or ex vivo. These may include but are not limited to, vascular endothelial growth factor, fibroblast growth factor (basic), bone morphogenetic protein 4, bone morphogenetic protein 7, insulin and its analogues, leptin, and growth hormone all at concentrations including, but not limited to, 1 to 1000 ng/ml.

By “plated at a density of between 100 to 100,000 cells per mm³” is meant a range of cell density expressed relative to the volume of the culture conditions. The values are so described to account for the fact that a three-dimensional tissue is being developed. These values represent a range only and final concentrations must be determined by those skilled in the art to achieve maximal adipocyte differentiation in the implants.

By “significantly” is meant statistically significant. In one nonlimiting embodiment, “significantly” refers to at least 2 or 3 percent and preferably at least 5 percent. In another preferred embodiment, “significantly” means at least 6, 8 or 10 percent.

II. Adipose-Derived Stem or Stromal Cells

Adipose-derived stem cells or “adipose-derived stromal cells” refer to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably the adipose tissue is human. A convenient source of adipose tissue is from liposuction surgery, however, the source of adipose tissue or the method of isolation of adipose tissue is not critical to the invention.

Adult human extramedullary adipose tissue-derived stromal cells represent a stromal stem cell source that can be harvested routinely with minimal risk or discomfort to the patient. Pathologic evidence suggests that adipose-derived stromal cells are capable of differentiation along multiple lineage pathways. Adipose tissue is readily accessible and abundant in many individuals. Obesity is a condition of epidemic proportions in the United States, where over 50% of adults exceed the recommended BMI based on their height.

It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal.

Adipose tissue offers many practical advantages for tissue engineering applications. First, it is abundant. Second, it is accessible to harvest methods with minimal risk to the patient. Third, it is replenishable. While stromal cells represent less than 0.01% of the bone marrow's nucleated cell population, there are up to 8.6×10⁴ stromal cells per gram of adipose tissue (Sen et al 2001, Journal of Cellular Biochemistry 81:312-319). Ex vivo expansion over 2 to 4 weeks yields up to 500 million stromal cells from 0.5 kilograms of adipose tissue. These cells can be used immediately or cryopreserved for future autologous or allogeneic applications.

Adipose derived stromal cells also express a number of adhesion and surface proteins. These include cell surface markers such as CD9; CD29 (integrin beta 1); CD44 (hyaluronate receptor); CD49d,e (integrin alpha 4, 5); CD54 (ICAM1); CD55 (decay accelerating factor); CD105 (endoglin); CD106 (VCAM-1); CD166 (ALCAM) and HLA-ABC (Class I histocompatibility antigen); and cytokines such as interleukins 6, 7, 8, 11; macrophage-colony stimulating factor; GM-colony stimulating factor; granulocyte-colony stimulating factor; leukemia inhibitory factor; stem cell factor and bone morphogenetic protein. Many of these proteins have the potential to serve a hematopoietic supportive function and all of them are shared in common by bone marrow stromal cells.

Methods for the isolation, expansion, and differentiation of human adipose tissue-derived cells have been reported. See for example, Burris et al 1999, Mol Endocrinol 13:410-7; Erickson et al 2002, Biochem Biophys Res Commun. Jan. 18, 2002; 290(2):763-9; Gronthos et al 2001, Journal of Cellular Physiology, 189:54-63; Halvorsen et al 2001, Metabolism 50:407-413; Halvorsen et al 2001, Tissue Eng. 7(6):729-41; Harp et al 2001, Biochem Biophys Res Commun 281:907-912; Saladin et al 1999, Cell Growth & Diff 10:43-48; Sen et al 2001, Journal of Cellular Biochemistry 81:312-319; Zhou et al 1999, Biotechnol. Techniques 13: 513-517. Adipose tissue-derived stromal cells are obtained from minced human adipose tissue by collagenase digestion and differential centrifugation [Halvorsen et al 2001, Metabolism 50:407-413; Hauner et al 1989, J Clin Invest 84:1663-1670; Rodbell et al 1966,. J Biol Chem 241:130-139]. Others have demonstrated that human adipose tissue-derived stromal cells can differentiate along the adipocyte, chondrocyte, and osteoblast lineage pathways [Erickson et al 2002, Biochem Biophys Res Commun. Jan. 18, 2002; 290(2):763-9; Gronthos et al 2001, Journal of Cellular Physiology, 189:54-63; Halvorsen et al 2001, Metabolism 50:407-413; Halvorsen et al, 2001, Tissue Eng. Dec. 7, 2001; (6):729-41; Harp et al 2001, Biochem Biophys Res Commun 281:907-912; Saladin et al 1999, Cell Growth & Diff 10:43-48; Sen et al 2001, Journal of Cellular Biochemistry 81:312-319; Zhou et al 1999, Biotechnol. Techniques 13: 513-517; Zuk et al 2001, Tissue Eng. 7: 211-228].

Human adipose tissue-derived adult stromal cells represent an adult stem cell source that can be harvested routinely with minimal risk or discomfort to the patient. They can be expanded ex vivo, differentiated along unique lineage pathways, genetically engineered, and re-introduced into individuals as either autologous or allogeneic transplantation.

WO 00/53795 to the University of Pittsburgh and The Regents of the University of California and U.S. patent application Ser. No. 2002/0076400 assigned to the University of Pittsburgh, disclose adipose-derived stem cells and lattices substantially free of adipocytes and red blood cells and clonal populations of connective tissue stem cells. The cells can be employed, alone or within biologically-compatible compositions, to generate differentiated tissues and structures, both in vivo and in vitro. Additionally, the cells can be expanded and cultured to produce hormones and to provide conditioned culture media for supporting the growth and expansion of other cell populations. In another aspect, these publications disclose a lipo-derived lattice substantially devoid of cells, which includes extracellular matrix material form adipose tissue. The lattice can be used as a substrate to facilitate the growth and differentiation of cells, whether in vivo or in vitro, into anlagen or mature tissue or structures. Neither publication discloses adipose tissue derived stromal cells that have been induced to express at least one phenotypic or genotypic characteristic of an intra-ocular stromal cell.

U.S. Pat. No. 6,391,297 assigned to Artecel Sciences discloses a composition of an isolated human adipose tissue-derived stromal cell that has been differentiated to exhibit at least one characteristic of an osteoblast that can be used in vivo to repair bone and treat bone diseases. This adipose-derived osteoblast-like cell can be optionally genetically modified or combined with a matrix.

U.S. Pat. No. 6,426,222 assigned to BioHoldings International discloses methods for inducing osteoblast differentiation from human extramedullary adipose tissue by incubating the adipose tissue cells in a liquid nutrient medium that must contain a glucocorticoid.

WO 00/44882 and U.S. Pat. No. 6,153,432 listing Halvorsen et al as inventors, discloses methods and compositions for the differentiation of human preadipocytes isolated from adipose tissue into adipocytes bearing biochemical, genetic, and physiological characteristics similar to that observed in isolated primary adipocytes.

WO 01/62901 and published U.S. patent application Ser. No. 2001/0033834 to Artecel Sciences discloses isolated adipose tissue-derived stromal cells that have been induced to express at least one phenotypic characteristic of a neuronal, astroglial, hematopoietic progenitor or hepatic cell. In addition, an isolated adipose tissue-derived stromal cell that has been dedifferentiated such that there is an absence of adipocyte phenotypic markers is also disclosed.

U.S. Pat. No. 6,429,013 assigned to Artecel Sciences discloses compositions directed to an isolated adipose tissue-derived stromal cell that has been induced to express at lease one characteristic of a chondrocyte. Methods are also disclosed for differentiating these cells.

U.S. Pat. No. 6,200,606 to Peterson et al. discloses that precursor cells which have the potential to generate bone or cartilage can be isolated from a variety of hematopoietic and non-hematopoietic tissues including peripheral blood, bone marrow and adipose tissue.

U.S. patent application Ser. No. 2000/00054331 to Fraser and Hedrick which was published Mar. 20, 2003 (filed Sep. 12, 2002) discloses an adipose-derived cell that is cryopreserved prior to subsequent therapeutic applications.

Zilberfarb et al. (J. Cell Science 110, 801-807, 1997), “Human Immortalized Brown Adipocytes Express Functional β₃-Adrenoreceptor Coupled to Lipolysis” discloses an immortalized cell line of human brown pre-adipocytes differentiated in culture into adipocytes that express the β₃-adrenoreceptor functionally coupled to adenylate cyclase and lipolysis.

The adipose tissue derive stromal cells useful in the methods of invention are isolated by a variety of methods known to those skilled in the art such as described in WO 00/53795 to the University of Pittsburgh et al. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject so as to provide for an autologous transplant. Alternatively, the transplanted cells are allogeneic.

The methods of the invention provide the distinct advantage of requiring few or no passes during culturing, thus increasing efficiency, increasing yield and decreasing cost.

As a non-limiting example, in one method of isolating adipose tissue derived stromal cells, the adipose tissue is treated with collagenase at concentrations between 0.01 to 0.5%, preferably 0.04 to 0.2%, most preferably 0.1%, trypsin at concentrations between 0.01 to 0.5%, preferably 0.04 to 0.04%, most preferably 0.2%, at temperatures between 25° to 50° C., preferably between 33° to 40° C., most preferably at 37° C., for periods of between 10 minutes to 3 hours, preferably between 30 minutes to 1 hour, most preferably 45 minutes. The cells are passed through a nylon or cheesecloth mesh filter of between 20 microns to 800 microns, more preferably between 40 to 400 microns, most preferably 70 microns. The cells are then subjected to differential centrifugation directly in media or over a Ficoll or Percoll or other particulate gradient. Cells can be centrifuged at speeds of between 100 to 3000×g, more preferably 200 to 1500×g, most preferably at 500×g for periods of between 1 minute to 1 hour, more preferably 2 to 15 minutes, most preferably 5 minutes, at temperatures of between 40° to 50° C., preferably between 20° to 40° C., most preferably at 25° C.

In yet another method of isolating adipose-derived stromal cells a mechanical system such as described in U.S. Pat. No. 5,786,207 to Katz et al is used. A system is employed for introducing an adipose tissue sample into an automated device, subjecting it to a washing phase and a dissociating phase wherein the tissue is agitated and rotated such that the resulting cell suspension is collected into a centrifuge-ready receptacle. In such a way, the adipose-derived cells are isolated from a tissue sample, preserving the cellular integrity of the desired cells.

III. Inducement of Adipose-Derived Stromal Cells to Exhibit at Least One Characteristic of an Adipocyte

The invention includes compositions comprising an adipose tissue derived stromal cell induced to form a cell that expresses at least one genotypic or phenotypic characteristic of an adipocyte which contains a substantially or significantly greater amount of extracellular matrix proteins than a mature isolated adipocyte. The differentiated cell also, or in the alternative, contains a substantially lower amount of lipid than a mature isolated adipocyte. The methods of the invention reliably provide a high differentiation frequency of isolated adipose-derived stem cells.

### ADVANTAGES OF THE DISCLOSED METHODS

Under the culture conditions disclosed herein, the differentiated cells exhibit multiple oil droplets. These oil droplets appear smaller in diameter than those found in either isolated mature adipocytes or differentiated adipocytes produces by any other method. Since lipid in the oil droplets is less dense than water, cells with smaller oil droplets, and therefore less lipid, are less likely to detach from cultureware and float to the surface of culture media. Cells that float are lost during subsequent feeding. In addition, adipocytes with larger oil droplets have a greater propensity to float during harvesting procedure, resulting in a loss of cells and reduced yield. Furthermore, adipocytes with small oil droplets are more resistant to mechanical damage or shearing force during cell harvesting and implanting procedures. These advantages can be demonstrated by measuring the integrity and yield of cells under these various conditions. The typical yield is greater than 90%. Due to host-to-host individual differences, the yield can be as low as 50%.

The culture procedures disclosed herein allow the cells to be continuously maintained in a single cultureware through proliferation and differentiation. The extracellular matrix proteins produced by the cells are accumulated without being digested during trypsinization and replating as described in other methods discussed above. As a consequence, the methods disclosed herein offer the distinct advantage of producing cells with more extracellular matrix proteins per unit of culture area. This is evident by the increased number of cells per culture area. The quantity of extracellular matrix proteins per unit of culture area can be determined by a variety of techniques, including but not limited to immunoassays.

The increased amount of extracellular matrix proteins provides another distinct advantage of the cells disclosed herein. Since the primary protein in extracellular matrix proteins is collagen which has been demonstrated to improve tissue cosmesis, cells produced by the methods disclosed provide an enhanced quantity of collagen when implanted into a host compared to isolated mature adipocytes or adipocytes produced by any other method.

Non-limiting examples of how to induce the differentiation of adipose-derived stromal cells include: 1) the use of cell media; 2) the use of support cells; 3) direct implantation of the undifferentiated cells into the tissue of a patient; and 4) cellular engineering techniques.

A) Cell Media Inducement

Treatment of the adipose-derived stromal cells with a medium containing a combination of serum, embryonic extracts, purified or recombinant growth factors, cytokines, hormones, and/or chemical agents, in a 2-dimensional or 3-dimensional microenvironment, will induce differentiation.

One non-limiting example of a method for differentiating an adipose-derived cells into a cell having a genotypic or phenotypic property of an adipocyte, comprises: plating isolated adipose-derived adult stem cells at a desired density, including but not limited to a density of about 1,000 to about 500,000 cells/cm²; incubating the cells in a chemically defined culture medium comprising at least one compound selected from the group consisting of: growth factor, hormone, cytokine, serum factor, nuclear hormone receptor agonist, or any other defined chemical agent.

More specifically, as a non-limiting example, the medium for differentiating adipose tissue-derived stem cells into adipocytes (hereinafter referred to as the “differentiation medium”) comprises a defined cell culture medium similar to that described [Ham R G 1963; Morton H J 1970; Dulbecco R 1959; Smith J K 1960; having or supplemented with 1000-4500 mg/liter glucose; a biological buffer; 0-100 μM biotin; 0-100 μM pantothenate; about 0.1 to 5 mM isobutylmethylxanthine; 10-1000 nM human insulin or an equivalent amount of an insulin analogue; about 10% to 0% fetal bovine serum; 10 nM to 1 μM of a glucocorticoid; and a concentration of a PPARγ agonist effective to stimulate differentiation of human stem cells, between 10 nM to 100 micromolar. The PPARγ agonist is not a thiazolidinedione. Preferably, the PPARγ agonist is indomethacin or an indomethacin derivative.

By a “defined cell culture medium” is meant a serum free, chemically defined cell growth medium. The medium may also contain biotin and pantothenate. Preferably the medium is Dulbecco's Modified Eagle Medium [Morton H J 1970; Dulbecco R 1959; Smith J K 1960] Ham's F-12 Nutrient Broth [Ham R G 1963; Morton H J 1970;] or Earl's medium [Earle W R 1943]; However, a variety of media, known to those skilled in the art, are useful in the methods of the invention.

Additional compounds may be included or added to the medium. For example, antibiotics, such as penicillin, streptomycin and fungizone are useful additives to the media of the invention.

The pH of the medium must be maintained during use, either through the inclusion of a biological buffer or by adjusting the CO₂ content in the atmosphere of the incubator. Preferably the medium is buffered by about NaHCO₃ and HEPES to a physiological pH.

Fetal bovine serum (FBS) is added to the defined cell culture medium at a concentration of about 0 to 10%.

By “equivalent amount of an insulin analogue” is meant an amount of a compound having the same biological activity in the cell cultures used in the invention, as does 10 nM to 1 μM human insulin. The compound may or may not be structurally related to insulin and may be synthetic, naturally occurring or recombinant.

By “glucocorticoid” is meant any steroid or steroid-like compounds capable of supporting cell growth and differentiation and functional derivatives thereof. Preferably, the glucocorticoid is dexamethasone, hydrocortisone or cortisol. Preferably the concentration of glucocorticoid is 16 nM to 1 μM; most preferably the concentration of glucocorticoid is about 1 μM.

By “PPARγ agonist” is meant a compound capable of activating the peroxisome proliferator-activated receptor gamma (PPARγ). Preferably the PPARγ agonist is indomethacin or any indomethacin derivative.

By “effective to stimulate the differentiation of an adipose tissue-derived stem cell” is meant having about the same effect on the ability to stimulate differentiation of adipose tissue-derived stem cells to adipocytes using to the methods of the invention, as does 0.5-1.0 μM BRL49653.

The methods of the invention utilize the above media to achieve a greater than 90% differentiation of cultured human stem cells into adipocytes. Thus, it is a further object of the invention to provide methods for differentiating human stem cells into adipocytes, comprising:

a) plating isolated human cells at a density of about 25,000 to 120,000 cells/cm² in a medium comprising a defined cell culture medium having or supplemented with 1000-4500 mg/liter glucose; a biological buffer; and about 0% to 10% fetal bovine serum (vol/vol);

b) incubating said cells at about 37° C. in about 5% CO₂ for 4-24 hours until said cells are about 95-100% confluent;

c) replacing said medium with a differentiation medium comprising a defined cell culture medium having or supplemented with 1000-4500 mg/liter glucose; 0-100 μM biotin, 0-100 μM pantothenate, a biological buffer; about 0.1 to 0.5 mM isobutylmethylxanthine; 10 nM to 1 μM human insulin; about 10% to 0% fetal bovine serum; 16 nM to 1 μM of a glucocorticoid; and a concentration of a PPARγ agonist effective to stimulate differentiation of a human stem cells, wherein the PPARγ is preferably indomethacin or an indomethacin derivative;

d) incubating said cells at about 37° C. in about 5% CO₂ for 3 days;

e) replacing said differentiation medium with an adipocyte medium comprising a cell culture medium having or supplemented with 1000-4500 mg/liter glucose; 0-100 μM biotin, 0-100μM pantothenate, a biological buffer; 10 μM to 1 μM human insulin; about 10% to 0% fetal bovine serum; 16 nM to 1 μM of a glucocorticoid;

f) incubating said cells at about 37° C. in about 5% CO₂ for about 7-20 days and refeeding said cells with said adipocyte medium every 3-4 days.

When initially plating stem cells in medium (step a), the cells must be plated at a density of 25,000-120,000 cells/cm². Preferably the cell density is greater than 30,000 cells/cm². Lower density plating of stem cells results in an overall lower differentiation percentage. When plated at a density of greater than 25,000 cells/cm² the stem cells are usually confluent after overnight incubation. If cells are not fully confluent at this point, they may be incubated for up to another 24 hours prior to refeeding with differentiation medium. Longer incubations prior to re-feeding result in a lower differentiation percentage.

Once the cells have been exposed to differentiation media (step c), they are susceptible to detaching from the plate if the media is either completely removed or quickly added.

Formation of oil droplets, a characteristic of adipocytes, will occur approximately 4 days after differentiation medium is added. However, there is some variability related to inter-patient variability and the site from which the stem cells were isolated. In general, greater than 90% of cells differentiate under the above conditions.

The disclosed methods offer the distinct advantage of culturing the cells in a single cultureware flask or container. Thus, the need for multiple cell passages and trypsin digestion to suspend the cells is completely eliminated, increasing both yield and quality of cells produced. A single cultureware flask or container also allows for longer growth periods, which facilitates the production of extracellular matrix proteins.

A variety of methods known to those skilled in the art may be used to determine the percentage of differentiated cells in vivo and ex vivo. Examples of such methods include those that assess biochemical or morphological characteristics, such as lipid deposits and adipocyte-specific proteins or mRNAs. In a preferred method, the cells or tissue are fixed in phosphate buffered formalin and stained with oil red O dye.

Media useful in the methods of the invention contain fetal serum of bovine or other species origin at a concentration of at least 1-10%. Embryonic extract of chicken or other species origin is present at a concentration of about 1% to 30%, preferably at least about 5% to 15%, most preferably about 10%.

By “growth factors, cytokines, hormones” is intended but not limited to the following specific factors including, but not limited to, growth hormone, erythropoeitin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogenetic protein at concentrations of between picogram/ml to milligram/ml levels. Additional components are optionally added to the culture medium. Such components include but are not limited to antibiotics, albumin, amino acids, and other components known to the art for the culture of cells. Additionally, components optionally are added to enhance the differentiation process. By “chemical agents” is meant steroids, retinoids, and other chemical compounds or agents that induce the differentiation of adipose derived stromal cells..

B) Use of Support Cells to Promote the Differentiation of the Adipose-Derived Stromal Cells

In another embodiment of the invention, support cells are used to promote the differentiation of the adipose-derived stromal cells prior to or following implantation into an animal host. The support cells can be human or non-human-animal derived cells. If non-human-animal support cells are used, the resulting differentiated cells are implanted via xenotransplantation.

Adipose-derived cells are isolated and cultured within a population of cells; most preferably the population is a defined population. The population of cells is heterogeneous and includes support cells for supplying factors to the cells of the invention. Support cells include other cell types which will promote the differentiation, growth and maintenance of the desired cells. As a non-limiting example, if an adipose-derived stromal cell that expresses at least one genotypic or phenotypic characteristic of an adipocyte which contains a substantially greater amount of extracellular matrix proteins and/or a substantially different amount of lipid than a mature isolated adipocyte is desired, adipose-derived stromal cells are first isolated by any of the means described above, and grown in culture in the presence of other support cells. For example, these support cells preferably possess the characteristic of adipose stromal cell types. In another embodiment, the support cells are derived from primary cultures of these cell types taken from cultured human organ tissue. In yet another embodiment, the support cells are derived from immortalized cell lines. In some embodiments, the support cells are obtained autologously. In other embodiments, the support cells are obtained allogeneically.

Support cells can also be genetically engineered to be support cells. The cells are genetically modified to express exogenous genes or to repress the expression of endogenous genes by any method described below or know to those skilled in the art.

C) Implantation

Adipose-derived stromal cells and differentiated cells expressing at least one genotypic or phenotypic characteristic of an adipocyte which contains a substantially or significantly greater amount of extracellular matrix proteins and/or a substantially lower amount of lipid than a mature isolated adipocyte that are useful in autologous and allogeneic transplantations are implanted into an animal. The differentiation takes place in vivo by means of factors naturally in the environment or introduced factors. In one embodiment, the site of transplantation is a diseased organ or tissue in need of cosmesis. In other embodiments the site of transplantation is subcutaneous, intraperitoneal, topical, intrasynovial, vaginal, rectal, or intrathecal. Preferably, the subject is mammalian, more preferably, the subject is human. The cell of the invention can be induced to differentiate in vitro or after implantation into a patient.

It is contemplated that when undifferentiated adipose-derived stromal cells are introduced into the subject, in one particular embodiment, they are introduced directly into a diseased organ or into the tissue in need of adipocytes. In yet another aspect of the invention, the undifferentiated adipose-derived stromal cells are introduced along with any of the support cells as described herein that will provide an environment suitable for the in vivo differentiation of the stromal cells. In another embodiment, the support cells are derived from primary cultures of these cell types. In yet another embodiment, the support cells are derived from immortalized cell lines. In some embodiments, the support cells are obtained autologously. In other embodiments, the support cells are obtained allogeneically.

In another embodiment, a dedifferentiated adipose-derived cell is provided in combination with a pharmaceutically acceptable carrier for a therapeutic application to an animal, including but not limited to tissue repair, regeneration, reconstruction or enhancement. Adipose-derived cells are cultured by methods such as disclosed in U.S. Pat. No. 6,153,432 to dedifferentiate the cells such that the dedifferentiated adult stem cells can then be induced to express genotypic or phenotypic characteristics of an adipocyte which contains a substantially greater amount of extracellular matrix proteins and/or a substantially lower amount of lipid than a mature isolated adipocyte. The dedifferentiated adipose-derived cells are modified to include a non-endogenous gene sequence for production of a desired extracellular matrix protein or peptide. The dedifferentiated adipose-derived cell can, in an alternative embodiment, be administered to a host in a two- or three-dimensional matrix for a desired therapeutic purpose. In one embodiment, the dedifferentiated cell is obtained autologously from the patient's own cells. Alternatively, the dedifferentiated cell is obtained allogeneically.

Encapsulation

The present invention provides a method for encapsulating the differentiated adipose-derived cells in a biomaterial compatible with transplantation into a mammal, preferably a human and then transplanting the encapsulated cells into an animal. The encapsulation material should be selected not hinder the release of desired proteins secreted by the adipose-derived adult stem cells. The materials used include but are not limited to collagen derivatives, hydrogels, calcium alginate, agarose, hyaluronic acid, poly-lactic acid/poly-glycolic acid derivatives and fibrin.

D) Genetic Manipulation of the Adipose-Derived Cells of the Invention

In yet another embodiment, the adipose-tissue derived cell expressing at least one genotypic or phenotypic characteristic of an adipocyte which contains a substantially or significantly greater amount of extracellular matrix proteins and/or a substantially or significantly lower amount of lipid than a mature isolated adipocyte is genetically modified to express exogenous genes or to repress the expression of endogenous genes and implanted into an animal. The invention provides a method of genetically modifying such cells and populations prior to implantation.

The cells of the invention can be modified to produce a greater amount of extracellular matrix protein, preferably wherein the extracellular matrix protein is collagen. Alternatively, the cells can be modified to produce a smaller amount of lipid or lipid-containing vacuoles.

A nucleic acid construct comprising a promoter and the sequence of interest can be introduced into a recipient prokaryotic or eukaryotic cell either as a non-replicating DNA (or RNA) molecule, which can either be a linear molecule or, more preferably, a closed covalent circular molecule. Since such molecules are incapable of autonomous replication without an origin of replication, the expression of the gene can occur through the transient expression of the introduced sequence. Alternatively, permanent expression can occur through the integration of the introduced DNA sequence into the host chromosome.

In one embodiment, a vector is employed which is capable of integrating the desired gene sequences into the host cell chromosome. Cells which have stably integrated the introduced DNA into their chromosomes can be selected by also introducing one or more markers which allow for selection of host cells which contain the desired nucleic acid sequence. The marker, if desired, can provide for prototrophy to an auxotrophic host, biocide resistance, e.g., resistance to antibiotics, or heavy metals, such as copper, or the like. The selectable marker gene sequence can either be directly linked to the DNA gene sequences to be expressed, or introduced into the same cell by co-transfection. Preferably, expression of the marker can be quantified.

In a preferred embodiment, the introduced nucleic acid molecule will be incorporated into a plasmid or viral vector capable of autonomous replication in the recipient host. Any of a wide variety of vectors can be employed for this purpose. Factors of importance in selecting a particular plasmid or viral vector include: 1) the ease with which recipient cells that contain the vector can be recognized and selected from those recipient cells which do not contain the vector; 2) the number of copies of the vector which are desired in a particular host; and 3) whether it is desirable to be able to “shuttle” the vector between host cells of different species.

Preferred eukaryotic vectors include but are not limited to, vaccinia virus, SV40, retroviruses, adenoviruses, adeno-associated viruses and a variety of commercially available, plasmid-based mammalian expression vectors that are familiar to those experienced in the art.

Once the vector or nucleic acid molecule containing the construct(s) has been prepared for expression, the DNA construct(s) can be introduced into an appropriate host cell by any of a variety of suitable means, i.e., transformation, transfection, viral infection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the heterologous protein.

Introduced DNA being “maintained” in cells should be understood as the introduced DNA continuing to be present in essentially all of the cells in question as they continue to grow and proliferate. That is, the introduced DNA is not diluted out of the majority of the cells over multiple rounds of cell division. Rather, it replicates during cell proliferation and at least one copy of the introduced DNA remains in almost every daughter cell. Introduced DNA may be maintained in cells in either of two fashions. First, it may integrate directly into the cell's genome. This occurs at a rather low frequency. Second, it may exist as an extrachromosomal element, or episome. In order for an episome not to be diluted out during cell proliferation, a selectable marker gene can be included in the introduced DNA and the cells grown under conditions where expression of the marker gene is required. Even in the case where the introduced DNA has integrated in the genome, a selectable marker gene may be included to prevent excision of the DNA from the chromosome.

The genetically altered cells can then be introduced into an organism by a variety of methods under conditions for the transgene to be expressed in vivo. As a non-limiting example, the transgene can encode for the production of an extracellular matrix protein, preferably wherein the transgene encodes for the production of collagen. The cells containing the transgene for the extracellular matrix protein can then be introduced into the animal. Alternatively, the cells containing the transgene are injected intraperitoneally or into some other suitable organ depot site.

E) Cellular Characterization

By “characterization” of the resulting differentiated cells is intended the identification of surface and intracellular proteins, genes, and/or other markers indicative of the lineage commitment of the stromal cells to a particular differentiated state. These methods can include, but are not limited to, (a) detection of cell surface proteins by immunofluorescent methods using protein specific monoclonal antibodies linked using a secondary fluorescent tag, including the use of flow cytometric methods; (b) detection of intracellular proteins by immunofluorescent methods using protein specific monoclonal antibodies linked using a secondary fluorescent tag, including the use of flow cytometric methods; (c) detection of cellular gene expression by polymerase chain reaction, in situ hybridization, and/or northern blot analysis; or (d) any other method know to those skilled in the art.

Adipocyte differentiated cells may be characterized by the identification of surface and intracellular proteins, genes, and/or other markers indicative of the lineage commitment of the stromal cells to a particular differentiated state. These methods, which are described above, include, but are not limited to, (a) detection of cell surface proteins by immunofluorescent assays such as flow cytometry or in situ immunostaining of adipose-derived stromal cells surface proteins such as CD36, lipoprotein lipase, and pref-1 in addition to those outlined in Gronthos et al 2001 (b) detection of intracellular proteins by immunofluorescent methods such as flow cytometry or in situ immunostaining of adipose tissue-derived stromal cells using specific monoclonal antibodies; (c) detection of the expression of lineage selective mRNAs such as but not limited to, adipocyte fatty acid binding protein aP2, leptin, lipoprotein lipase, and adipsin by methods such as polymerase chain reaction, in situ hybridization, and/or other blot analysis (See Gimble et al. 1989 Blood 74:303-311).

F) Use of the Cells of the Invention as Therapeutic Agents

The adipose-derived cells and populations described herein can be employed as therapeutic agents in animals, for example, in tissue cosmesis or repair and in diseases requiring the addition of fat cells such as lipodystrophy. Generally, such methods involve transferring the cells to the desired tissue or depot. The cells are transferred to the desired tissue by any method appropriate, which generally vary according to the tissue type. For example, cells can be transferred to a graft by bathing the graft or infusing it with culture medium containing the cells. Alternatively, the cells can be seeded on the desired site within the tissue to establish a population. Cells can be transferred to sites in vivo using devices well know to those skilled in the art for example, catheters, trocars, cannulae, or stents seeded with the cells.

III. Tissue Engineering

Adipose-derived cells can be isolated and differentiated into a cell that possesses at least one characteristic of an adipocyte which contains a substantially greater amount of extracellular matrix proteins and/or a substantially different amount of lipid than a mature isolated adipocyte and then engineered into tissue matter, tissues or organs to be implanted into an animals. The tissue matter can include, for example a portion of, or even a whole organ. As such, prior to implantation into an animal, the cells described herein are used in combination with any known technique of tissue engineering, including but not limited to those technologies described in the following: U.S. Pat. Nos. 5,902,741 and 5,863,531 to Advanced Tissue Sciences, Inc.; U.S. Pat. No, 6,139,574, Vacanti et al.; U.S. Pat. No. 5,759,830, Vacanti et al.; U.S. Pat. No. 5,741,685, Vacanti,; U.S. Pat. No. 5,736,372, Vacanti et al.; U.S. Pat. No. 5,804,178, Vacanti et al.; U.S. Pat. No. 5,770, 417, Vacanti et al.; U.S. Pat. No. 5,770,193, Vacanti et al.; U.S. Pat. No. 5,709,854, Griffith-Cima et al.; U.S. Pat. No. 5,516,532, Atala et al.; U.S. Pat. No. 5,855,610, Vacanti et al.; U.S. Pat. No. 5,041,138, Vacanti et al.; U.S. Pat. No. 6,027,744, Vacanti et al.; U.S. Pat. No. 6,123,727, Vacanti et al.; U.S. Pat. No. 5,536,656, Kemp et al.; U.S. Pat. No. 5,144,016, Skjak-Braek et al.; U.S. Pat. No. 5,944,754, Vacanti; U.S. Pat. No. 5,723,331, Tubo et al.; and U.S. Pat. No. 6,143,501, Sittinger et al..

To produce such a structure, the cells and populations are maintained under conditions suitable for them to expand and divide to form the organ. This may be accomplished by transferring them to an animal typically at a sight at which the new matter is desired. Thus, the invention can facilitate the regeneration of tissue within an animal where the cells are implanted into such tissues.

In still other embodiments, the cells are induced to differentiate and expand into tissue in vitro prior to implantation into an animal. As such, the cells are cultured on substrates that facilitate formation into three-dimensional structures conducive for tissue development. Thus, for example, the cells are cultured or seeded onto a bio-compatible lattice, such as one that includes extracellular matrix material, synthetic polymers, cytokines, growth factors, etc. Such a lattice can be molded into desired shapes for facilitating the development of tissue types. The lattice can be formed from polymeric material, having fibers as a mesh or sponge. Such a structure provides sufficient area on which the cells can grow and proliferate. Desirably, the lattice is biodegradable over time, so that it will be absorbed into the animal matter as it develops. Suitable polymeric lattices can be formed from monomers such as glycolic acid, lactic acid, propyl fumarate, caprolactone, and the like. Other lattices can include proteins, polysaccharides, polyhydroxy acids, polyorthoesters, polyanhydrides, polyphosphozenes, or synthetic polymers, particularly biodegradable polymers, or any combination thereof. Also, the lattice can include hormones, such as growth factors, cytokines, morphogens (e.g. retinoic acid etc), desired extracellular matrix materials (e.g. fibronectin, laminin, collagen etc) or other materials (e.g. DNA, viruses, other cell types etc) as desired.

The cells are introduced into the lattice such that they permeate into interstitial spaces therein. For example, the matrix can be soaked into a solution or suspension containing the cells, or they can be infused or injected in the matrix. Preferably, a hydrogel formed by cross-linking of a suspension including the polymer and also having the inventive cells dispersed therein is used. This method of formation permits the cells to be dispersed throughout the lattice, facilitating more even permeation of the lattice with the cells. Of course, the composition also can include support cells for supplying factors to the cells of the invention. Support cells include other cell types which will promote the differentiation, growth and maintenance of the adipocyte cells.

Those skilled in the art will appreciate that lattices suitable for inclusion into the implanted material can be derived from any suitable source, e.g. Matrigel™, and can of course include commercial sources for suitable lattices. Another suitable lattice can be derived from the acelluar portion of adipose tissue for example adipose tissue extracellular matrix substantially devoid of cells. Typically such adipose-derived lattices include proteins such as proteoglycans, glycoproteins, hyaluronin, fibronectins, collagens and the like, all of which serve a excellent substrates for cell growth. Additionally, such adipose-derived lattices can include hormones, cytokine, growth factors and the like. Those skilled in the art would be aware of methods for isolating such an adipose-derived lattice such as that disclosed in WO 00/53795 to the University of Pittsburgh.

In yet another embodiment of the invention, tissue is created using solid free-form fabrication methods to allow for tissue regeneration and growth for implantation into an animal. Such techniques are disclosed, for example, in U.S. 6,138,573 to Vacanti et al and allow the creation of partial or whole organs for implantation into a human in need thereof. Creation of such partial or whole organs is accomplished with the cells of the present invention obtained in an autologous manner. Alternatively, such partial or whole organs are created from cells of the invention that were obtained in an allogeneic manner. It is contemplated that any method known to those skilled in the art is useful for engineering tissue from the cells of the invention. As a non-limiting example, U.S. Pat. Nos. 6,022,743 and 5,516,681 to Naughton et al (Advanced Tissue Sciences) disclose methods for 3-dimensional cell culture systems for the culture of pancreatic-like tissue.

Such techniques could easily be adapted for other types of tissue cosmesis or repair, for example, the construction and repair of breasts following radical or partial mastectomy or for breast augmentation surgery. These techniques involve the seeding and implanting of cells onto a matrix to form organ tissue and structural components which can additionally provide controlled release of bioactive agents. The matrix is characterized by a network of lumens functionally equivalent to the naturally occurring vasculature of the tissue formed by the implanted cells and which is further lined with endothelial cells. The matrix is further coupled to blood vessels or other ducts at the time of implantation to form a vascular or ductile network throughout the matrix. The free-form fabrication techniques refer to any technique know in the art that builds a complex 3-dimensional object as a series of 2-dimensional layers. The methods can be adapted for use with a variety of polymeric, inorganic and composite materials to create structures with defined compositions, strengths and densities. Thus, utilizing such methods, precise channels and pores can be created within the matrix to control subsequent cell growth and proliferation within the matrix of one or more cells types having a defined function. In such a way, differentiated adipose-derived cells, corresponding to the various types of a particular organ's cells can be combined to form a partial or whole organ. Such cells are combined in the matrix to provide a vascular network lined with endothelial cells interspersed throughout the cells. Other structures can also be formed for use as lymph ducts, bile and other exocrine or excretory ducts within the organ.

The cells, populations, lattices and compositions used in the methods of the invention are used in tissue engineering and regeneration in animals. Thus, the invention pertains to the use of an implantable structure incorporating any of the disclosed inventive features. The exact nature of the implant will vary according to the use desired. The implant can comprise mature tissue or can include immature tissue or the lattice. Thus for example, an implant can comprise a population of cells that are undergoing differentiation, optionally seeded within a lattice of a suitable size and dimension. Such an implant is injected or engrafted within an immune compromised host to encourage the generation or regeneration of mature tissue within the animal.

The adipose-derived lattice is conveniently employed as part of a cell culture kit for use in animals. Accordingly, the invention provides a kit including adipose-derived lattice and one or more other components, such as hydrating agents (e.g. water, physiologically-compatible saline solutions, prepared cell culture media, serum or combinations or derivatives thereof), cell culture substrates (e.g. dishes, plates vials etc), cell culture media (whether in liquid or powdered form), antibiotics, hormones and the like. While the kit can include any such ingredients, preferably it includes all ingredients necessary to support the culture and growth of the desired cells upon proper combination for use in an animal. The desired kit can also include cells which are seeded into the lattice as described.

IV. Use of the Invention in Immune-Compromised Animals

In one embodiment of the invention, an adipose-derived stem cell is induced to express at least one phenotypic characteristic of an adipocyte which contains a substantially greater amount of extracellular matrix proteins and/or a substantially lower amount of lipid than a mature isolated adipocyte that is then implanted into an immune compromised animal of a different species. In a further embodiment of the invention, an adipose-derived stem cell is directly implanted into an immune compromised animal of a different species by any of the methods described above.

V. Screening of Therapeutic Agents

The disclosed cells can also be utilized for the screening and characterization of therapeutic agents for efficacy and toxicity in a variety of diseases in which fat cells or fat cell metabolism is critical. Such diseases include but are not limited to obesity, diabetes, cardiovascular disease as well as diseases in which altered lipolysis and lipogenesis play a role. Compounds can be identified and studied that enhance or inhibit the differentiation of adipose tissue derived stem cells into adipocytes.

The effects of potential therapeutic agents can be tested in animals bearing tissues and organs generated from adipose-derived stem cells. For example, a therapeutic agent may be any known agent having a therapeutic effect on a target cell, such effect being selected from, but not limited to: correcting a defective gene or protein, a drug action, a toxic effect, a growth stimulating effect, a growth inhibiting effect, a metabolic effect, a catabolic affect, an anabolic effect, an antiviral effect, an antibacterial effect, a hormonal effect, a neurohumoral effect, a cell differentiation stimulatory effect, a cell differentiation inhibitory effect, a neuromodulatory effect an antineoplastic effect, an insulin stimulating or inhibiting effect, a bone marrow stimulatory effect, a pleuripotent stem cell stimulating effect, an immune system stimulating effect, and/or any other known therapeutic effects that may be provide by a therapeutic agent administered to a animal model according to the present invention.

The present invention now is described more fully by the following examples. This invention may, however, be embodied in many different forms and should not be construed as limited to the embodiments set forth herein; rather, these embodiments are provided so that this disclosure is thorough and complete, and fully conveys the scope of the invention to those skilled in the art.

### EXAMPLES

### Example 1

### Isolation, Expansion and Differentiation of Human Adipose Tissue-Derived Stem Cells

Human adipocyte differentiation has previously been induced within 24 hours after stromal cells were plated. The pre-differentiated stromal cells were in passage 1 or passage 2. We have demonstrated that cells can be cultured for growth and differentiation in one cultureware without the need for cell passage. By eliminating cell passage, the manufacturing process can be streamlined and costs can be reduced.

The differentiation of human adipose tissue-derived stem cells from a single donor under two growth conditions was examined. In the two arms of the study, the primary isolated adipose tissue-derived stem cells were inoculated into individual cultureware vessels (A and B) and maintained in culture for 12 days until confluence. At confluence, cells from vessel A were harvested by trypsin digestion and replated in a second cultureware vessel as “Passage 1” (P1). In contrast, the cells in vessel B were maintained in culture without trypsin digestion and were maintained as “Passage 0” (P0). On day 13 of the study, both sets of cultures (P0 and P1) were exposed to differentiation inductive media outlined in the accompanying tables:

| | | |
|---|---|---|
| Stock (mM) | FAC^{a} (μM) | Volume needed |

Differentiation Medium I

| | | | | |
|---|---|---|---|---|
| DME/H12 | | | 50 | ml |
| FBS | 100% | 3% | 1.5 | ml |
| IBMX | 200 | 250 | 62.5 | μl |
| BRL49653 | 10 | 1 | 5 | μl |
| Dexamethasone | 20 | 1 | 2.5 | μl |
| Insulin | 1 | 0.1 | 5 | μl |
| Biotin | 66 | 33 | 25 | μl |
| Pantothenate | 100 | 17 | 8.5 | μl |
| DMSO | 100% | 0.1% | 45 | μl |

Differentiation Medium II

| | | | | |
|---|---|---|---|---|
| DME/H12 | | | 50 | ml |
| FBS | 100% | 3% | 1.5 | ml |
| IBMX | 200 | 250 | 62.5 | μl |
| Indomethacin | 100 | 100 | 50 | μl |
| Dexamethasone | 20 | 1 | 2.5 | μl |
| Insulin | 1 | 0.1 | 5 | μl |
| Biotin | 66 | 33 | 25 | μl |
| Pantothenate | 100 | 17 | 8.5 | μl |

FAC^{a}: Final Assay Concentration

Cells were induced with the two distinct differentiation media for 4 days and then converted to a common adipocyte media. Cells were monitored by morphologic examination and for secretion of the adipocyte-derived cytokine, leptin, over the next 7 to 10 days. The degree of lipid accumulation within the cells was determined by direct visual examination with or without oil red O staining.

The level of leptin secretion was determined by ELISA assay using a commercially available protocol (R&D Systems, Minneapolis Minn.). Visual inspection indicated that the overall degree of lipid accumulation in cultures differentiated at P0 was as good or better than that observed in cells induced at P1. Leptin ELISA assays of cells at day 7 post differentiation demonstrated that values from P0 (828+/−71 pg/ml) were comparable to P1 (689+/−44 pg/ml).

These findings indicate that the adipose tissue-derived stem cells can be differentiated along the adipocyte lineage pathway without passaging them among multiple cultureware containers. This improvement has significant commercial and scientific benefits for anyone practicing this invention and skilled in the art.

### Example 2

### Improved Production of Extracellular Matrix Proteins by Differentiating Human Adipose Tissue-Derived Stem Cells

The improved culture methods for isolation, expansion and differentiation of human adipose tissue-derived stem cells outlined in Example 1 results in a product with superior characteristics that facilitate soft tissue cosmesis and tissue repair. This is evident in the expression levels of extracellular matrix proteins by the differentiated cells, including but not limited to, aggrecan, type I collagen, type IV collagen, integrins, hyaluronate, proteoglycans, and other cell adhesion molecules (CAMs). The human adipose tissue-derived stem cells cultured under the methods in Example 1 can be monitored for the expression of these extracellular matrix proteins by a number of methods known to those skilled in the art, including but not limited to: (a) flow cytometric analysis of the adipose tissue-derived stem cells in suspension with monoclonal antibodies directed against selected extracellular matrix proteins [Gronthos et al 2001]; (b) enzyme linked immunoabsorbant assays (ELISA) of the differentiated adipose tissue-derived stem cells and/or a protein extract thereof with antibodies directed against selected extracellular matrix proteins; (c) radioisotopic labeling of adipose tissue-derived stem cell-synthesized proteins or proteoglycans using [¹⁴C] amino acids and/or [³⁵S] sulfate [Erickson et al 2002]. The improved culture methods result in increased expression levels of extracellular matrix proteins and proteoglycans as compared to previous methodologies developed by the inventors [Halvorsen et al 2001; Sen et al 2001] and reported by others [Hauner et al 1989].

### Example 3

### Improved Production of Differentiated Human Adipocytes for Clinical Applications

The improved culture methods for expansion and differentiation of human adipose tissue-derived stem cells outlined in Example 1 results in a product with superior characteristics that facilitate soft tissue cosmesis and tissue repair. This is evident in the smaller size of oil droplets accumulated in each adipocyte compared to either primary adipocytes or adipocytes differentiated using previous methods [Halvorsen et al 2001 ]. The quantity of lipid accumulation can be determined by a number of methods known to those skilled in the art, including but not limited to: (a) determination of quantity of triglyceride using an enzymatic assay (lipase acting on triglyceride to produce glycerol; glycerol kinase acting on glycerol to produce glycerol 3 phosphate; glycerol phosphate oxidase acting on glycerol 3 phosphate to release peroxide; peroxidase acting on peroxide in the presence of chemical substrate to produce calorimetric reagent for quantification; Sigma Infinity™ Triglycerides Reagent); (b) quantifying lipid accumulation by microscopic image analysis (Mashiba, et al, 2001). The improved method of producing adipocytes involves cells with higher density when differentiation is induced. In addition, the differentiation is induced with indomethacin, a PPARγ ligand that is less lipogenic than many thiazolidinediones. Combining these two improvements, the resulting adipocytes can be harvested and concentrated completely with moderate centrifugal force, resulting in higher cell number yields than in previously described methods. The cells are also less likely to be sheared or damaged during harvesting and injection, which will be critical in clinical applications.

### Example 4

### Generation of Three Dimensional Adipose Tissue Ex Vivo

Stem cells are isolated from human subcutaneous adipose tissue according to methods described in Example 1 above. The cells are plated at a density of 500 to 120,000 cells per cm². After reaching confluence, the cells are converted to a differentiation medium containing insulin, biotin, pantothenate, indomethacin, dexamethasone, and isobutylmethylxanthine. After an additional three to five days in culture in this medium, the cells are harvested by trypsin/EDTA digestion at 37° C. for a period of 1 to 15 minutes, and suspended in an equal volume of medium containing a minimum of 1% fetal bovine serum. The cells are centrifuged at 300×g for 5 minutes at 20° C. The concentrated cells are resuspended in Adipocyte Maintenance Medium containing glucose, 3% fetal bovine serum, insulin, biotin, pantothenate, and dexamethasone. The cells are resuspended at a concentration between 100,000 to 1 million per ml. The concentrated cells are pipetted directly onto a poly lactic-co-glycolic polymer disk of porosity 50% to 95%, preferably at 90%, with a thickness between 1 to 10 mm, most preferably 2.5 mm, and a diameter of 5 to 25 mm, most preferably 12.5 mm, with a pore size range of 50 to 1000 mum, most preferably 200 to 600 mum. Matrices can be synthesized with the biomaterial alone or with incorporated adipocyte inducing materials. These include, but are not limited to, adipogenic compounds (such as the thiazolidinedione BRL49653, indomethacin or indomethacin derivatives, growth hormone, dexamethasone, basic fibroblast growth factor, and/or MAP Kinase inhibitors), vascularization compounds (such as monobutyrin), and/or expression vectors containing cDNAs encoding adipogenic factors (such as constitutively active peroxisome proliferator activated receptor gamma2 or constitutively active bone morphogenetic protein receptor IA) or vascularization inducing factors (such as vascular endothelial growth factor). Alternatively, cells are resuspended in Matrigel or in another alternative biocompatible material such as alginate at concentrations between 100,000 to 1 million per ml; these may also incorporate additional factors as described above. The cells in the 3-dimensional matrix are maintained in adipocyte maintenance medium continuously and replaced with fresh medium every 2 to 4 days, most preferably every third day. Cell differentiation along the adipocyte lineage is monitored by the appearance of lipid vacuoles based on phase contrast microscopy and by staining with oil red O. The 3-dimensional matrices can be examined directly by light microscopy or embedded in paraffin for sectioning. Additional methods to monitor adipogenesis include detection of adipocyte specific gene markers such as, but not limited to, adipocyte fatty acid binding protein aP2, leptin, lipoprotein lipase, and adipsin by northern blot, western blot, ELISA, and PCR analyses. These methods can be used to optimize conditions for adipocyte differentiation ex vivo and to determine the length of time permitted for maximal adipogenic commitment prior to implantation or in vivo studies.

### Example 5

### Generation of Implantable Three-Dimensional Adipose Tissue Depots In Vivo Using Immunodeficient Rodent Models—In Vivo Differentiation

The three-dimensional stromal cell matrices developed in Example 3 above are employed for in vivo implantation. Immunodeficient rodent models include, but are not limited to, severe combined immunodeficient (SCID) mice, nude mice, nude/beige mice, SCID/non-obese diabetic (NOD) mice, and nude rats. Two methods are described below but these are not exclusive of alternative approaches. In the first method, harvested stem cells are maintained in culture for no more than one passage to obtain maximal numbers of cells.

Stem cells are isolated from human subcutaneous adipose tissue according to methods described in Example 1 above. The cells are plated at a density of 500 to 120,000 cells per cm². The undifferentiated stem cells are harvested by trypsin/EDTA digestion at 37° C. for a period of 1 to 15 minutes, and suspended in an equal volume of medium containing a minimum of 10% fetal bovine serum. The cells are centrifuged at 100 to 500×g, preferably at 282×g for 1 to 10 minutes, preferably for 5 minutes at 4° C. to 37° C., preferably at 20° C. The concentrated cells are resuspended in adipocyte maintenance medium containing 1000 to 10,000 mg glucose per liter, preferably 3150 mg/liter, 1 to 10% fetal bovine serum, preferably 3% fetal bovine serum, insulin, biotin, pantothenate, and dexamethasone at concentrations known to those skilled in the art. The cells are resuspended at a concentration between 1000 to 10 million cells per ml, more preferably at 100,000 to 1 million per ml. The concentrated cells are pipetted directly onto a poly lactic-co-glycolic polymer disk of porosity 50% to 95%, preferably at 90%, with a thickness between 1 to 10 mm, most preferably 2.5 mm, and a diameter of 5 to 25 mm, most preferably 12.5 mm, with a pore size range of 50 to 1000 mum, most preferably 200 to 600 mum.

Matrices can be synthesized with the biomaterial alone or with incorporated adipocyte inducing materials. These include, but are not limited to, adipogenic compounds (such as the thiazolidinedione BRL49653, indomethacin or indomethacin derivatives, growth hormone, dexamethasone, basic fibroblast growth factor, and/or MAP Kinase inhibitors), vascularization compounds (such as monobutyrin), and/or expression vectors containing cDNAs encoding adipogenic factors (such as constitutively active peroxisome proliferator activated receptor gamma2 or constitutively active bone morphogenetic protein receptor IA) or vascularization inducing factors (such as vascular endothelial growth factor) Alternatively, cells are resuspended in Matrigel or in another alternative biocompatible material such as alginate at concentrations between 100,000 to 1 million per ml; these may also incorporate additional factors as described above.

### Example 6

### Generation of Implantable Three-Dimensional Adipose Tissue Depots In Vivo Using Immunodeficient Rodent Models—In Vitro Differentiation

Alternatively, the cells are permitted to initiate adipocyte differentiation prior to incorporation into the three dimensional matrix. Stem cells are isolated from human subcutaneous adipose tissue according to methods described in Example 1 above. The cells are plated at a density of 500 to 120,000 cells per cm². After reaching confluence, the cells are converted to “Adipocyte Differentiation Medium” containing insulin, biotin, pantothenate, indomethacin, dexamethasone, and isobutylmethylxanthine or equivalent compounds at concentrations known to those skilled in the art. After an additional three days in culture in this medium, the cells are harvested by trypsin/EDTA digestion at 37° C. for a period of 1 to 15 minutes, and suspended in an equal volume of medium containing a minimum of 10% fetal bovine serum. The cells are centrifuged at 100 to 500×g, preferably at 300×g for 1 to 10 minutes, preferably for 5 minutes at 4° C. to 37° C., preferably at 20° C. The concentrated cells are resuspended in Adipocyte Maintenance Medium containing 1000 to 10,000 mg glucose per liter, preferably 4500 mg/liter, 1 to 10% fetal bovine serum, preferably 3% fetal bovine serum, insulin, biotin, pantothenate, and dexamethasone at concentrations known to those skilled in the art. The cells are resuspended at a concentration between 1000 to 10 million cells per ml, more preferably at 100,000 to 1 million per ml. The concentrated cells are pipetted directly onto a poly lactic-co-glycolic polymer disk of porosity 50% to 95%, preferably at 90%, with a thickness between 1 to 10 mm, most preferably 2.5 mm, and a diameter of 5 to 25 mm, most preferably 12.5 mm, with a pore size range of 50 to 1000 mum, most preferably 200 to 600 mum.

Matrices can be synthesized with the biomaterial alone or with incorporated adipocyte inducing materials. These include, but are not limited to, adipogenic compounds (such as the thiazolidinedione, indomethacin, an indomethacin derivative, growth hormone, dexamethasone, basic fibroblast growth factor, and/or MAP Kinase inhibitors), vascularization compounds (such as monobutyrin), and/or expression vectors containing cDNAs encoding adipogenic factors (such as constitutively active peroxisome proliferator activated receptor gamma2 or constitutively active bone morphogenetic protein receptor IA) or vascularization inducing factors (such as vascular endothelial growth factor). Alternatively, cells are resuspended in Matrigel™ or in another alternative biocompatible material such as alginate at concentrations between 100,000 to 1 million per ml; these may also incorporate additional factors as described above.

Prior to implantation, cells can be marked by exposure to adenoviral vectors expressing the green fluorescent protein, beta-galactosidase, or other marker protein or enzyme, by exposure to retroviral vectors expressing the same markers, by exposure to fluorescent probes, or by other standard or newly developed methodologies. These methods permit the identification of the donor cells in the host recipient animal at later time.

The resulting three-dimensional matrices are implanted in one of the immunodeficient rodent models described above. Animals are anesthetized with ketamine and rompine or an equivalent anesthetic/analgesic by intraperitoneal injection or a veterinarian approved and reviewed alternative administration method. Implants are maintained for periods of 1 day to 12 months, more preferably 3 weeks to 12 weeks, most preferably for 5 weeks. Animals are fed a regular chow diet (4-5% fat), a high fat diet (10⁻³⁰% fat, either omega-3 or omega-6 enriched), a high carbohydrate diet (>50% carbohydrate), or a high fat/high carbohydrate diet during part or all of this period. The presence of human adipocytes in the animals is detected during this period by collection of serum and ELISA assay for the human form of the adipocyte specific hormone, leptin. At the conclusion of the study, implants are harvested by surgical removal and analyzed by histochemical, immunofluorescent, biochemical, and molecular biological techniques for the appearance of adipocytes or fat cells in the implant site. The presence of differentiated human adipocytes is determined by detection of the unique human DNA gene marker, the “alu” fragment, using in situ PCR methods.

In addition, methods to detect any marker proteins, enzymes or fluorescent probes are utilized to document the presence of donor cells in the final differentiated implant. The cellular composition, size, and viability of the implant are determined at this time. These methods are used to optimize the growth conditions, factors, proteins, cDNAs, and biomaterials necessary to support adipocyte differentiation by the donor human stem cells in the host animal.

This approach can be modified to prepare a selective modeled three-dimensional implant. The biomaterial can be shaped to meet specifications required for a particular need. To test this approach, biocompatible polymers are prepared with varying widths, heights and thickness to determine the ability to create “designer” soft tissue depots. The degree of these tests in rodents may be limited. Alternative large animal models (dogs, pigs, sheep) will be considered to test the dimensional limits facing this tissue engineering approach. The volume ratio of the tissue depot may correlate with the actual size of the host animal and may not reflect the geometry of the implant itself.

### Example 7

### Generation of Injectable Three-Dimensional Adipose Tissue Depots In Vivo Using Immunodeficient Rodent Models

The three-dimensional stromal cell matrices developed in Example 3 above are employed for in vivo implantation. Immunodeficient rodent models include, but are not limited to, severe combined immunodeficient (SCID) mice, nude mice, nude/beige mice, SCID/non-obese diabetic (NOD) mice, and nude rats. Two methods are described below but these are not exclusive of alternative approaches. In the first method, harvested stem cells are maintained in culture for no more than one passage to obtain maximal numbers of cells. Stem cells are isolated from human subcutaneous adipose tissue according to methods described in Example 1 above. The cells are plated at a density of 500 to 120,000 cells per cm². The undifferentiated stem cells are harvested by trypsin/EDTA digestion at 37° C. for a period of 1 to 15 minutes, and suspended in an equal volume of medium containing a minimum of 10% fetal bovine serum. The cells are centrifuged at 100 to 500×g, preferably at 282×g for 1 to 10 minutes, preferably for 5 minutes at 4 ° C. 37° C., preferably at 20° C. The concentrated cells are resuspended in adipocyte maintenance medium containing 1000 to 10,000 mg glucose per liter, preferably 3150 mg/liter, 1 to 10% fetal bovine serum, preferably 3% fetal bovine serum, insulin, biotin, pantothenate, and dexamethasone at concentrations known to those skilled in the art. The cells are resuspended at a concentration between 1000 to 10 million cells per ml, more preferably at 100,000 to 1 million per ml, most preferably at 2 million cells per ml.

In the second method, the cells are permitted to initiate adipocyte differentiation prior to incorporation into the three dimensional matrix as described in Example 6. Stem cells are isolated from human subcutaneous adipose tissue according to methods described in Example 1 above. The cells are plated at a density of 500 to 20,000 cells per cm². One day after plating, the cells are converted to “Adipocyte Differentiation Medium” containing insulin, biotin, pantothenate, indomethacin, dexamethasone, and isobutylmethylxanthine or equivalent compounds at concentrations known to those skilled in the art. After an additional three to five days in culture in this medium, the cells are harvested by trypsin/EDTA digestion at 37° C. for a period of 1 to 15 minutes, and suspended in an equal volume of medium containing a minimum of 10% fetal bovine serum. The cells are centrifuged at 100 to 500×g, preferably at 282×g for 1 to 10 minutes, preferably for 5 minutes at 4° C. to 37° C., preferably at 20° C. The concentrated cells are resuspended in adipocyte maintenance medium containing 1000 to 10,000 mg glucose per liter, preferably 3150 mg/liter, 1 to 10% fetal bovine serum, preferably 3% fetal bovine serum, insulin, biotin, pantothenate, and dexamethasone at concentrations known to those skilled in the art. The cells are resuspended at a concentration between 1000 to 10 million cells per ml, more preferably at 100,000 to 1 million per ml, most preferably at 2 million cells per ml.

Prior to implantation, cells can be marked by exposure to adenoviral vectors expressing the green fluorescent protein, beta-galactosidase, or other marker protein or enzyme, by exposure to retroviral vectors expressing the same markers, by exposure fluorescent probes, or by other standard or newly developed methodologies. These methods permit the identification of the donor cells in the host recipient animal at later time.

The resulting cells from either method are mixed with liquid Matrigel™ (collagen type IV) or other liquid biocompatible polymer at a cell concentration of between 50,000 to 5 million cells per ml, most preferably at one million cells per ml, and a Matrigel™ concentration of 5 to 20 mg per ml, most preferably at 10 mg per ml. The suspensions can be synthesized with the biomaterial alone or with incorporated adipocyte inducing materials. These include, but are not limited to, adipogenic compounds (such as the thiazolidinedione indomethacin, an indomethacin derivative, growth hormone, dexamethasone, basic fibroblast growth factor, and/or MAP Kinase inhibitors), vascularization compounds (such as monobutyrin), and/or expression vectors containing cDNAs encoding adipogenic factors (such as constitutively active peroxisome proliferator activated receptor gamma2 or constitutively active bone morphogenetic protein receptor IA) or vascularization inducing factors (such as vascular endothelial growth factor) The cell/Matrigel™ suspension is then injected into one of the immunodeficient rodent models described above. Prior to injection, animals are anesthetized with ketamine and rompine or an equivalent anesthetic/analgesic by intraperitoneal injection or a veterinarian approved and reviewed alternative administration method. Studies are conducted with the review and approval of an institutional animal care and utilization committee. Implants are maintained for periods of 1 day to 12 months, more preferably 3 weeks to 12 weeks, most preferably for 5 weeks. Animals are fed a regular chow diet (4-5% fat), a high fat diet (10⁻³⁰% fat, either omega-3 or omega-6 enriched), a high carbohydrate diet (>50% carbohydrate), or a high fat/high carbohydrate diet during part or all of this period. The presence of human adipocytes in the animals is detected during this period by collection of serum and ELISA assay for the human form of the adipocyte specific hormone, leptin. At the conclusion of the study, implants are harvested by surgical removal and analyzed by histochemical, immunofluorescent, biochemical, and molecular biological techniques for the appearance of adipocytes or fat cells in the implant site. The presence of differentiated human adipocytes is determined by detection of the unique human DNA gene marker, the “alu” fragment, using in situ PCR methods, or other methods known to those skilled in the art.

In addition, methods to detect any marker proteins, enzymes or fluorescent probes are utilized to document the presence of donor cells in the final differentiated implant. The cellular composition, size, and viability of the implant are determined at this time. These methods are used to optimize the growth conditions, factors, proteins, cDNAs, and biomaterials necessary to support adipocyte differentiation by the donor human stem cells in the host animal.

Many modifications and other embodiments of the invention come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. An adipose tissue-derived adult stem cell that is differentiated to express at least one characteristic of an adipocyte and which contains a substantially greater amount of extracellular matrix proteins than a mature isolated adipocyte.

2. An adipose tissue-derived adult stem cell that is differentiated to express at least one characteristic of an adipocyte and which contains a significantly greater amount of extracellular matrix proteins than a mature isolated adipocyte.

3. An adipose tissue-derived adult stem cell that is differentiated to express at least one characteristic of an adipocyte and which contains a substantially smaller amount of lipid than a mature isolated adipocyte.

4. An adipose tissue-derived adult stem cell that is differentiated to express at least one characteristic of an adipocyte and which contains a significantly smaller amount of lipid than a mature isolated adipocyte.

5. The cell of claim 1, wherein the extracellular matrix protein is collagen.

6. The cell of claim 2, wherein the extracellular matrix protein is collagen.

7. The cell of any of claims 1-6, wherein the cell is human.

5. The cell of any of claims 1-6, wherein the cell is modified with a nucleic acid.

6. The cell of any of claims 1-6, wherein the cell is modified with a chemical probe.

7. A method for expanding the growth of an isolated adipose tissue-derived stem cell comprising:
(a) plating the cells in a single container at varying densities in a growth maintenance medium comprising a chemically defined cell culture medium without enzymatic digestion and re-plating of the cells; and
(b) incubating the cells for a growth period to optimize the production of extracellular matrix.

8. A method for differentiating an adipose tissue-derived stem cell into a cell that possesses at least one characteristic of an adipocyte, comprising:
(a) plating the cells at varying densities in a single container in a growth maintenance medium comprising a chemically defined cell culture medium without enzymatic digestion and re-plating of the cells;
(b) incubating the cells for a cell growth period to optimize the production of extracellular matrix;
(c) replacing the growth maintenance medium with an adipocyte differentiation medium comprising a defined cell culture medium having or supplemented with a concentration of a peroxisome proliferator-activated receptor gamma (PPARγ) agonist that is not a thiazolidinedione
(d) incubating the cells;
(e) replacing the adipocyte differentiation medium with a growth maintenance medium;
(f) incubating the cells; and
(g) harvesting the cells

9. A method for differentiating an adipose tissue-derived stem cell into a cell that possesses at least one characteristic of an adipocyte, comprising:
(a) plating the cells at varying densities in a single container in a growth maintenance medium comprising a chemically defined cell culture medium without enzymatic digestion and re-plating of the cells;
(b) replacing the growth maintenance medium with an adipocyte differentiation medium comprising a defined cell culture medium having or supplemented with a concentration of a peroxisome proliferator-activated receptor gamma (PPARγ) agonist that is not a thiazolidinedione
(c) incubating the cells;
(d) replacing the adipocyte differentiation medium with a growth maintenance medium;
(f) incubating the cells for a growth period to optimize the production and size of lipid vacuoles; and
(g) harvesting the cells

10. A method for differentiating an adipose tissue-derived stem cell into a cell that possesses at least one characteristic of an adipocyte, comprising:
(a) plating the cells at varying densities in a single container in a growth maintenance medium comprising a chemically defined cell culture medium without enzymatic digestion and re-plating of the cells;
(b) incubating the cells for a cell growth period to optimize the production of extracellular matrix;
(c) replacing the growth maintenance medium with an adipocyte differentiation medium comprising a defined cell culture medium having or supplemented with a concentration of a peroxisome proliferator-activated receptor gamma (PPARγ) agonist that is not a thiazolidinedione
(d) incubating the cells;
(e) replacing the adipocyte differentiation medium with a growth maintenance medium;
(f) incubating the cells for a growth period to optimize the production and size of lipid vacuoles; and
(g) harvesting the cells

11. A method for developing a three dimensional biomaterial matrix containing the adipose tissue-derived stem cells of any of claims 1-4, wherein the cells are capable of generating an adipose tissue depot upon implantation into a host recipient.

12. The method of claim 11, wherein the biomaterial matrix is selected from the group consisting of include poly-lactic acid, poly-glycolic acid, alginate, and a collagen type derivatives.

13. The method of claim 12, further comprising a chemical inducing factor.

14. The method of claim 13, wherein the chemical inducing factor comprises a protein, lipid, carbohydrate, polypeptide, nucleic acid or hormone.

15. The method of claim 13, wherein the chemical inducing factor enhances the adherence, growth, differentiation, proliferation, vascularization and three-dimensional modeling of adipose tissue-derived stem cells into a soft tissue or adipose tissue depot either in vivo or ex vivo.

16. The method of claim 13, wherein the chemical inducing factor comprises monobutyrin, a thiazolidinedione, a glucocorticoid, or long chain fatty acid.

17. The method of claim 13, wherein the chemical inducing factor comprises indomethacin or an indomethacin derivative.

18. The method of claim 13, wherein the chemical inducing factor is at a concentration of 10⁻⁹ to 10⁻³ M.

19. The method of claim 12, further comprising an exogenous growth factor.

20. The method of claim 19, wherein the exogenous growth factor comprises a cytokine, vascular endothelial growth factor, fibroblast growth factor (beta), bone morphogenetic protein 4, bone morphogenetic protein 7, insulin, an insulin analogue, leptin, or growth hormone.

21. The method of claim 19, wherein the exogenous growth factor is at a concentration of 1 to 1000 ng/ml.

22. The cell of any of claims 1-6 implanted into an immunodeficient rodent.

23. The cell of any of claims 1-6 further comprising a label with a probe.

24. The cell of claim 23, wherein the probe is adenoviral, retroviral, or fluorescent.

25. The cell of any of claims 1-6, implanted into a host.

26. The cell of claim 25 wherein the host is in need of tissue repair.

27. The cell of claim 26, wherein the host in need of tissue cosmesis.

28. The cell of any of claims 1-6, that is allowed to differentiate in vivo.

29. A differentiated adipose-tissue derived cell that contains a substantially greater amount of collagen than a mature isolated adipocyte.

30. A differentiated adipose-tissue derived cell that contains a significantly greater amount of collagen than a mature isolated adipocyte.

31. The cell of claim 29 that is used in tissue cosmesis.

32. The cell of claim 30 that is used in tissue cosmesis.

33. The cell of claim 29 that is used in tissue repair.

34. The cell of claim 30 that is used in tissue repair.
